(19) United States
(12) Patent Application Publication
(10) Pub. No.: US 2026/0294610 A1
(43) Pub. Date: Oct. 1, 2026

(54) SUBSTANCE DELIVERY DEVICE FOR DEEP DELIVERY OF EMBRYOS OR SEMEN IN THE UTERINE HORN OF A SOW OR A GILT

(71) Applicant: Norsvin SA, HAMAR (NO)

(72) Inventors: Bjørn GULBRANDSEN, HAMAR (NO); Reina JOCHEMS, HAMAR (NO); Louisa Jane ZAK, BEUNINGEN (NL); Camilla OLSTAD KRISTIANSEN, HAMAR (NO)

(73) Assignee: Norsvin SA, HAMAR (NO)

(21) Appl. No.: 19/481,592

(22) PCT Filed: May 10, 2024
(86) PCT No.: PCT/EP2024/062970

§ 371 Date: Nov. 5, 2025

(30) Foreign Application Priority Data
May 11, 2023 (NL) 2034805

(51) Int. Cl. A61D 19/02; A61D 19/04; A61M 25/00

(52) U.S. Cl. CPC A61D 19/027; A61D 19/04; A61M 25/0041

(57) ABSTRACT

Substance delivery device for deep delivery of embryos or semen in the uterine horn of a pig, in particular a sow or a gilt, comprising an introduction probe to be partially introduced into the cervix of the pig, provided with a guide lumen; and a delivery catheter, slidably arranged in the guide lumen, comprising: a proximal end and a distal end an inlet port arranged near the proximal end and a delivery lumen extending from the inlet port towards the distal end, a delivery section arranged at or near the distal end to be introduced inside the uterine horn, comprising a delivery opening fluidly connected to the delivery lumen, and a tip element, arranged at the distal end; wherein the tip element comprises a bend in a bending direction that is less than 45° to steer the delivery catheter, and wherein the delivery opening is positioned with respect to the bending direction to allow fluid flow out of the delivery opening, wherein a length of the delivery catheter from the proximal end up to the delivery opening is at least 1.7 m, such that upon insertion of the introduction probe in the cervix, the delivery catheter may be advanced further into the reproductive tract passing the cervix and uterine body and entering the uterine horn for delivery fluid comprising embryos and/or semen into the uterine horn through the delivery opening.

### FIELD OF THE INVENTION

The present invention relates to a substance delivery device for deep delivery of embryos or semen in the uterine horn of a pig, a substance delivery assembly, and to a method for delivery of embryos or semen.

### BACKGROUND OF THE INVENTION

Due to the anatomical complex structure of the pig reproductive tract, introduction of embryos or semen, or artificial insemination (AI) is relatively difficult. In particular, inserting a catheter through the cervix and into the uterine horns is a challenging task. Amongst others, complicating factors are the relatively narrow opening of the cervix with the cervical folds, the relatively sharp bend of approximately 90 degrees ventrally near the uterine horns, and, in particular, the length of the uterine horns and their tortuous shape.

At present, catheters for post-cervical artificial insemination or intra-uterine insemination are known, the first one allows to deposit semen in the uterine body and the second one makes it possible to pass the cervix for depositing semen in the uterine horns. This is of interest, as the semen dose can be reduced without impairing reproductive performance compared to conventional AI where semen is deposited in the cervix.

However, it has been found that even with the known intra-uterine AI-catheter, navigation of the catheter remains difficult, such that the catheter can be advanced into the uterine horn up to a limited extent only.

Depending on the background of the specific pig, such as breeding method, breed of pig and possible undergone surgery, a different insemination procedure and/or a different type of catheter may have been required.

US2004162461A1 discloses a method and apparatus for non-surgical embryo transfer or artificial insemination of mammals, comprising a sheath having a frusto-conical rearward end provided with a depositing chamber having an aperture to permit depositing into the uterus.

US2002193658A1 discloses an internal catheter that is advanced from a foam or spiral exterior catheter through the cervix, such that the tip of the internal catheter can be positioned into the uterine body or possibly into one of the uterine horns.

US2002038113A1 discloses an inner sheath-distal probe end combination for atraumatic navigation and entering the uterine body.

Conventional catheter systems as described above have several disadvantages and have so far been applied in sows and/or gilts at limited efficiency. As a result, obtained results in terms of pregnancy and depositing success rate leave room for improvement.

As an alternative, a surgical procedure may be performed, such as utero-tubal junction insemination using laparoscopy. However, surgery requires special medical expertise and may involve health risks to the pig.

### OBJECT OF THE INVENTION

It is therefore an object of the invention to provide a substance delivery device for deep delivery of embryos and/or semen in the uterine horn of a pig that overcomes the disadvantages of the prior art, or at least to provide an alternative delivery device, for example delivery device which enables delivery deep in the uterine horn, close to the utero-tubal junction, and/or which allows to use a highly lowered number of sperm cells without compromising success rate.

### SUMMARY OF THE INVENTION

The present invention provides a substance delivery device for deep delivery of embryos or semen in the uterine horn of a pig, in particular a sow or a gilt according to claim 1. The substance delivery device comprises an introduction probe to be partially introduced into the cervix of the pig, provided with a guide lumen; and a delivery catheter, slidably arranged in the guide lumen.

The delivery catheter comprises a proximal end and a distal end, an inlet port arranged near the proximal end and a delivery lumen extending from the inlet port towards the distal end, a delivery section arranged at or near the distal end to be introduced inside the uterine horn, comprising a delivery opening fluidly connected to the delivery lumen and a tip element, arranged at the distal end.

The delivery section comprises a tip element arranged at the distal end, which comprises a bend in a bending direction that is less than 45° to steer the delivery catheter, and wherein the delivery opening is positioned with respect to the bending direction to allow fluid flow out of the delivery opening, such that upon insertion of the introduction probe in the cervix, the delivery catheter may be advanced further into the reproductive tract passing the cervix and uterine body and entering the uterine horn for delivery of embryos and/or semen into the uterine horn through the delivery opening.

The length of the delivery catheter from the proximal end up to the delivery opening is at least 1.7 m.

The advantageous combination of the introduction probe and delivery catheter having the relatively long length allows for an efficient introduction deeply in the reproductive tract for advancing the delivery catheter tip deeply into the uterine horn for deep delivery of embryos or semen.

With the substance delivery device, the cervix and the uterine horn of a sow or a gilt may be navigated to deposit embryos and/or semen into the uterine horn closer to the place of ovulation than in the prior art. In particular, it may now advantageously be possible to pass the cervix more easily and insert the catheter very deep into the uterine horns, for example close to the utero-tubal junction. This way, the number of sperm cells required in a semen dose may be reduced, which is of considerable importance when using sex sorted semen or semen of interest. Also, embryos may advantageously be delivered deep in the uterine horn, such as at the tip of the uterine horn to achieve a successful pregnancy. Thus, the success rate of pregnancy may be improved. In addition, the substance delivery device may be suitable for deep delivery of other substances, such as other fluids.

The delivery catheter may be configured to be advanced through the cranial opening of the cervix when the introduction tube is inserted partially into the cervix and not through the cranial opening of the cervix.

By introducing the delivery catheter with the introduction probe, the cervix may be passed in a relatively short time. It has been found that with the delivery device, relatively high cervical pass ratios may be achieved of up to 100% in a single attempt. As such, the procedure can be performed relatively quickly and is relatively advantageous for animal welfare.

The tip element may be advanced into a selected uterine horn and may be configured to steer the delivery catheter into and through the selected uterine horn.

The delivery catheter may be relatively torsion stiff such that the tip element may be rotated by rotating a proximal end of the catheter, such that a uterine horn may be selected by rotation of the tip element.

As the tip element is shaped to steer the delivery catheter, a relatively good manoeuvrability may be achieved without guide wire. Therewith, a guide wire or other support element for constricting movement freedom of the tip element in the uterine horn may potentially be omitted.

The delivery catheter may be positioned to perform delivery at a desired depth in the uterine horn, for example deep in the uterine horn, such as near the utero-tuberal junction. The delivery catheter may be advanced in the uterine horn up to a greater or lesser depth. Therefore, the delivery catheter may be used in multiple circumstances, for example when the pig has abnormalities in the reproductive tract, when the pig has undergone surgery, and/or has differences in the reproductive tract due to aging or earlier litter(s) of the pig. This allows the delivery device to be used for different pig breeds, in different countries and according to different breeding methods.

Having a relatively good manoeuvrability, a relatively good animal welfare can be achieved with less or possibly even completely without any anaesthetic, such that animal wellbeing can potentially be improved, and the use of medication can therefore potentially be reduced.

Therewith, the delivery of embryos and/or semen can be carried out without substantial health risks to the pig. As a result, even no signs of discomfort were observed during in vivo tests.

By delivery of embryos and/or semen deep in the uterine horn, pregnancy can be induced and/or the chances can at least be improved. Further embryos and/or semen may be delivered for pig breeding and/or for scientific research.

The introduction probe may, with a distal end thereof, be configured to be positioned in the cervix of the pig to introduce the delivery catheter in the cervix.

The introduction probe may comprise a proximal end and a distal end and extend therebetween. The proximal end of the introduction probe may comprise a manipulator and/or a grip for manipulation by an operator.

In addition or alternatively, the proximal end of the delivery catheter may comprise a manipulator and/or a grip for manipulation by an operator.

The distal end may comprise a catheter opening through which the delivery catheter may be advanced to extend out of the catheter opening, for example into the cervix and/or the uterus of the pig.

The introduction probe may be narrowing towards the distal end thereof. Additionally, or alternatively, the introduction probe may have a rounded and/or smooth shape towards the distal end. These features may ease introduction of the introduction probe.

The introduction probe may, towards the distal end, have a decreasing diameter. An end surface of the introduction probe at the distal end may be rounded. A rounded end and/or a decreasing diameter may assist to gradually find a way through the caudal opening and/or along the cervical folds.

The introduction probe may be relatively rigid, such that the anatomy of the reproductive tract may be felt by the operator. In particular, the introduction tube may have a higher stiffness than the delivery catheter, such as a higher bending stiffness and a higher longitudinal stiffness.

The introduction probe may be straight. A straight introduction probe may help an operator to introduce the introduction probe in the reproductive tract, in particular to move the introduction probe through the caudal opening and past the cervical folds.

The introduction probe may have a flat outer surface. The outer surface may be a smooth surface. By having a flat and/or smooth outer surface, friction forces between the outer surface and the reproductive tract may be reduced.

A length of the introduction probe may, for example, be at least 30 cm, such as at least 35 cm. This way, the proximal end of the introduction probe may be manipulated while the distal end is introduced into the cervix, in particular through the caudal opening but not through the cranial opening thereof.

The introduction probe may comprise a holding element to hold the delivery catheter during introduction of the introduction probe. The holding element may hold the delivery catheter at a fixed position in the guide lumen. The holding element may be provided nearby the distal end of the introduction probe, for example in or adjacent to the guide lumen.

The introduction probe may be configured to be positioned, from the perspective of an operator, in front of the caudal opening of the cervix. The introduction probe may be relatively stiff to allow an operator to feel the position of the introduction probe with respect to the tissue of the cervix, such that the introduction probe may be inserted partially into the cervix, for example through the caudal opening but not through the cranial opening thereof.

Therewith, the introduction probe may provide external support to the delivery catheter in the cervix, such that excessive bending of the delivery catheter can be avoided. Furthermore, the introduction probe provides space through which the delivery catheter may be advanced relatively easily compared to advancing a catheter without introduction probe, for example a catheter having a guide wire.

It has been found that partial insertion of the introduction probe is sufficient for guiding the delivery catheter through the cervix. Further, by not having to pass through the cranial opening with the introduction probe, the procedure may be performed relatively fast and the risk of damaging the uterus with the introduction probe is reduced.

The delivery catheter is slidably arranged in the guide lumen of the introduction probe and is configured to be introduced in the cervix with the introduction probe. Upon insertion of the introduction probe, the delivery catheter may be advanced to extend out of the introduction probe into the cervix and/or the uterus of the pig. The delivery catheter may for example be configured to extend through an opening in communication with the guide lumen at the distal end of the introduction probe.

The guide lumen may follow the shape of the introduction probe. The guide lumen may be relatively straight. The guide lumen may be provided with a smooth inner surface.

An inner side of the guide lumen may have a relatively low friction coefficient. For example, the inner side may be provided with a friction-reducing coating. Additionally or alternatively, an outer side of the delivery catheter may be provided with a relatively low friction coefficient, for example be provided with a friction-reducing coating. An inner diameter of the guide lumen may be larger than a diameter of narrow passages in the cervix, e.g. larger than a distance between side walls of the cervix in a natural state. These features have been found to ease sliding of the delivery catheter through the guide lumen.

The delivery catheter comprises a tip element shaped to steer the catheter, such that the delivery catheter may pass the cervix and uterine body into an uterine horn. In particular, the delivery catheter may pass the cervix, uterine body and/or uterine horn without being supported by the introduction tube through the entire cervix.

The tip element comprises a bend in a bending direction. The angle of the bend is less than 45°. It has been found that this way, the delivery catheter may be steered into a selected uterine horn. The bend may bias the catheter in a biasing direction, for example by rotating the catheter such that the tip element points towards one of the uterine horns, further advancement of the catheter may result in the catheter entering the respective one of the uterine horns.

The tip element may be autoclave-resistant. In addition to the tip element, the delivery section, the delivery catheter and/or the introduction probe may partially or entirely be autoclave-resistant. The tip element may be autoclave-resistant for at least 15, such as 30, minutes at 121° C., and/or at least 3, such as 6, minutes at 134° C., for example at a steam pressure of 2-5 bar. By having autoclave-resistance, safe reuse of the catheter and/or introduction probe may be possible.

The tip element, the delivery section and/or the delivery lumen may have a smooth surface. By having a smooth surface, adhesion of substance, such as embryos or semen, on the surface may be reduced, such that the chances of damage thereof are lowered.

The delivery opening is positioned with respect to the bending direction to allow fluid flow out of the delivery opening. For example, the delivery opening may be arranged through an outer surface of the delivery section. The delivery opening may be arranged at a distance from the tip element, or in the tip element.

A single delivery opening may be arranged in the delivery catheter. This, way, the delivery location may be controlled relatively precisely.

Alternatively, multiple delivery openings may be arranged on the delivery section, for example evenly distributed over the delivery section or grouped together, e.g. spaced at a distance from each other as described above. By having multiple openings, the substance may be delivered at multiple points in the uterine horn.

The delivery openings may be spaced evenly around the circumference at a respective cross section of the delivery section, such that even delivery in of the uterine horn may be possible.

Delivery deep in the uterine horn may be performed by providing a substance via the delivery lumen and the delivery opening. The delivery opening may have a diameter of equal to a diameter of the delivery lumen, or another diameter providing for gradual widening or narrowing of a delivery fluid flow.

The substance to be delivered may be a fluid. The fluid may be a liquid, such as a buffered saline solution. The fluid may comprise an embryo culture medium.

In an embodiment, the delivery opening is arranged in the elongate element and/or in the tip element. By providing the delivery fluid nearby the distal end of the delivery catheter, delivery may be performed deep in the uterine horn.

An outer diameter of the delivery catheter may be less than 10 mm, for example less than 8 mm. This outer diameter has been found to allow for sufficient torsional and longitudinal stiffness for sufficient steerability and pushability of the catheter, while still providing sufficient elasticity to follow the uterine horns. It has been found that a smaller diameter is advantageous for delivery of substances, in particular semen and/or embryos, as it allows to reach deeply into the uterine horn.

In an embodiment, the tip element is a rounded cone. The tip element may be an oblique cone having a base plane and a top. The cone may have a rounded top.

In an embodiment, the tip element is a bent rounded cone. The tip element may be bent having the cone axis in a the bending direction.

The cone is rounded, i.e. the tip of the cone is not a sharp tip. The cone tip may be fully rounded, i.e. be semi-spherical, partially rounded and/or the cone may be frustoconical, i.e. have a partially flattened tip. The tip of the cone may have a radius of less than 8 mm, for example less than 4 mm, most preferably less than 3 mm. This way, the tip may be advanced into the uterine horn relatively well while reducing the chances of tissue damage.

The bending direction may also be referred to as the first bending direction. The tip element may comprise a bend having an angle of 2°-30°, preferably an angle of 6-8°. It has been found that these angles are especially advantageous for manoeuvring into and through the uterine horn.

In a further embodiment, the tip element may comprise a double bent. For example, the tip element may be bent in a first bending direction and in a second bending direction. The tip element may be bent in the second bending direction at an angle that is less than 4°, in particular less than 25°, for example 5°-10° with respect to a longitudinal axis of the delivery section.

The top may also be biased in a second bending direction, such that a projection of the top on the base plane is not located in the centre of the plane.

As such, the tip element is biased in the first and/or second bending direction and, when advanced against a transverse surface, the delivery catheter is steered in the respective bending direction, such that by orienting the tip element around its longitudinal axis, a position of the rounded cone with respect to uterine horns may be selected. This way, the first and/or second bending direction may be chosen to coincide with one of the uterine horns.

In an embodiment, no collar is provided around the circumference of the catheter between the tip element and the delivery opening. The catheter may have a smooth outer surface between the tip element and the delivery opening. It has been found that this reduces friction with tissue and/or the introduction probe.

In an embodiment, the delivery catheter, between the tip element and the guide lumen, is free from outer protrusions such as collars and/or cuffs. The delivery catheter may be free from outer protrusions between the proximal end and the inlet port. As such, friction may be reduced further.

In another embodiment, the catheter comprises a collar arranged near the tip element and/or the delivery section. The collar may protrude from the circumference of the catheter, for example radially as a ring. The collar may improve feedback to an operator, e.g. by enhancing the feeling of forces exerted between the catheter and the reproductive tract.

In an embodiment, a distance between the tip element and the delivery opening is less than 50 cm, such as less than 30 cm, in particular less than 10 cm.

By having a relatively small distance, substance is delivered near the tip element, i.e. advantageously deep in the uterine horn. Further, delivery near the tip element minimises contact with the catheter to limit the amount of delivered substance that is slid back away from the desired delivery position when removing the catheter.

In an embodiment, a distance between the delivery catheter comprises an uteral section and wherein the tip element and/or the delivery section are distinct elements coupled to the uteral section. The uteral section may be configured to, during delivery, extend through the uterine body.

The uteral section may extend between the proximal end and the delivery section. The uteral section may be manufactured separately from the tip element and/or the flushing section.

The uteral section may comprise a different material than the tip element and/or the flushing section. The uteral section may comprise a tube and/or a hose. The uteral section may be opaque and/or transparent.

It has been found that advantageously, the uteral section may efficiently be produced separately, for example be cut from a reel, from the tip element and/or the delivery section, which may for example be cast, printed or milled.

The uteral section and the tip element and/or the delivery section may be rigidly connected to each other, for example by means of interlocking, threads or glue. The tip element and the delivery section may be one element, or be distinct from each other.

The tip element and/or the delivery section may be coupled to the uteral section via a connection. The connection may be a threaded connection, friction connection, snap fit connection, other types of connection or combinations thereof. The connector may enable usage of commonly available catheter parts to be connected to the tip element and/or the delivery section to ease production of the delivery catheter. Additionally or alternatively, the connector may allow the catheter to be adapted to the specific animal.

In an embodiment, the tip element and/or the delivery section are detachably coupled to the uteral section. Detachably coupling may allow for replacement and/or sterilisation of parts of the catheter. Also, this way, the catheter may easily be adapted to the specific pig.

Additional or alternative connections may be arranged between other parts of the catheter, e.g. by having connectors arranged therebetween.

The delivery section may comprise a connector, such as a screw thread connection, friction connector, snap fit connector, other connector or combinations thereof. A complementary connector adapted to cooperate with the connector of the delivery section may be arranged on the rest of the catheter having additional elongate elements. A commonly available catheter part may be provided with the complementary connector, or the complementary connector may be arranged therein, e.g. by milling.

The connector may comprise radial protrusions that are non-rotationally symmetric This way, rotation forces may be transferred via the connector. As such, rotation between the connector and a complementary connector may be reduced or avoided to improve steering of the delivery catheter. For example, the radial protrusions may provide the connector with a non-circular cross sectional shape, such as an oval shape.

The connector may comprise an indent having a first diameter and one or more ridges having a second diameter larger than the first diameter. The ridges may provide additional outer surface to the connector which may improve connection strength, e.g. by having a larger surface for friction and/or glue. Additionally or alternatively, the connector may connect by having a complementary connector snapping around the indent behind a ridge, or vice versa.

In an embodiment, the delivery catheter may partially or entirely be made of a silicone material, such as silicone rubber. Silicone rubber has been found not react with many substances to be delivered and not to be toxic for the embryos and/or semen.

Relatively convenient steerability of the delivery catheter may achieved by having a relatively high torsional stiffness to allow for rotation around a longitudinal axis, and a relatively low bending stiffness for following the shape of the uterine horn. Thereby, the introduction tube may prevent early bending of the delivery catheter with the guide lumen. The steerability of the catheter may be achieved by the bent tip element. Further, outer diameter of the catheter, diameter of the delivery lumen and/or the material used may contribute to the steerability, for each of these properties, advantageous values are explained herein.

Pushability of the delivery catheter may be ensured by having a relatively high longitudinal stiffness to be advanced through the cranial opening of the cervix and into the uterine horn.

The tip element may be made from silicone rubber, for example silicone rubber having an elastic modulus of 2.5-7.5 N/mm2, such as TrueSil A50, which provides flexibility for advancement through the uterine horns. Such materials have been found to provide an advantageous combination of steerability and pushability.

In an embodiment, the tip element is connected to the flushing section and/or to the uteral section via a glue, in particular a silicone glue, such as Dow Corning 782. A silicone glue has similar advantages as the silicone material and may also be well suited to glue silicone.

In an embodiment, the tip element and/or the flushing section may have an elastic modulus of 2.5-7.5 N/mm2. The tip element may be less rigid than the flushing section and/or an elongate element. This way, the chances of the delivery catheter getting stuck in the reproductive tract may be reduced due to elasticity of the tip element. Further, a relatively flexible tip element allows the tip element to bend into curves for advantageous steering of the delivery catheter

In an embodiment, the tip element comprises a material having an A classification of A35-A100, such as A50-A100. The A classification may also be referred to as the Shore A hardness scale, i.e. a Shore A hardness scale of 35-100, such as 50-100. It has been found that the hardness of materials having this hardness are suited for reaching deep into the uterine horn.

In an embodiment, the delivery section comprises a material having an A classification of A35-A100, such as A50-A100. This delivery section hardness may further improve reaching deep into the uterine horn. It has been found that the delivery opening should not be too soft in order to avoid deformation that could block delivery of substance.

In an embodiment, a diameter of the delivery lumen is at least 1 mm, for example 1 2.5 mm. A diameter of the delivery lumen may for example be 1-6 mm, for example 1-2 mm or 2.5-3.5 mm. A relatively low diameter may be advantageous as it allows to limit the amount of substance, e.g. fluid, that needs to be used for delivery deep in the uterine horn. On the other hand, a very low diameter lumen may increase required pressure for pumping the substance through the delivery lumen.

An inner side of the delivery lumen may have a relatively low friction coefficient. For example, the inner side may be provided with a friction-reducing coating. The inner side of the delivery lumen may be hydrophobic. Having a low-friction and/or hydrophobic surface may ease delivering substance through the delivery lumen.

In an embodiment, the delivery section comprises an elongate element that spaces the tip element from the uteral section in a longitudinal direction, for example wherein a length from the tip element to the uteral section is 5-70 cm, for example 5-50 cm. By having a larger distance, the delivery section may be larger, such that substance may be delivered over a larger part of the uterine horn. E.g., by having a relatively large delivery opening and/or multiple delivery openings. However, by having a smaller distance, the delivery catheter may be employed in a larger variety of uterine horns.

Additionally, it has been found that delivery deep in the uterine horn yields most successful results. A distance of 5-30 cm has been found to provide the highest yield.

The delivery catheter may be configured to deliver substances without additional pushing and/or pulling wire for delivery of substances by means of mechanical pushing and/or pulling action.

In an embodiment, a longest length of the substance delivery device, e.g. including the parts introduced into the pig, is 1.7 m.

In an embodiment, a length of the delivery catheter from the proximal end up to the delivery opening is in the range of 170 to 345 cm. It has been found that these lengths may be sufficient for deep delivery in the uterine horn and thereby provide a favourable balance between sufficient length and practical handling of the catheter

In an embodiment, the delivery catheter may have a length from the proximal end up to the delivery opening of at least 2.0 meter, for example at least 2.5 meter.

The invention further relates to a substance delivery assembly, comprising a substance delivery device according to any of the embodiments disclosed herein; an injection device, such as a syringe or pump, fluidly connectable to the delivery lumen via the inlet port for injecting deliverable fluid in the uterine horn through the delivery opening; and a an injectable substance container, e.g. a fluid container, associated with the injection device and fluidly connectable to the delivery lumen for deep delivery of embryos or semen in the uterine horn. The assembly allows the delivery of substances with benefits similar to those described hereinabove.

In an embodiment, the delivery assembly comprises an imaging system, such as an ultrasound or pulsating doppler system, for imaging a position of the tip element with respect to the uterine horn. Using an imaging system, the position of the tip element may be determined relatively precisely such that delivery may be performed in a specific part of the uterine horn. In particular, the position of the tip element deep in the uterine horn may be verified.

In an embodiment, the fluid container comprises a deliverable fluid, wherein the deliverable fluid comprises embryos and/or semen, for example wherein the volume of the deliverable fluid is less than 600 μL. As the substance is delivered deep, a relatively low amount of substance may be required.

The fluid container may comprise a fluid having a relatively high viscosity. For example, the viscosity may be higher than a viscosity of a flushing fluid used for flushing embryos in the uterine horn of a pig.

In an embodiment, the injection device is configured to inject the deliverable fluid by injecting a transport fluid before and/or after the deliverable fluid. It has been found that this way, the amount of substance may be reduced, as pressure may be provided by the transport fluid. The transport fluid may be a gas, such as air. In use, the substance may be pushed through the delivery lumen by the transport fluid.

The invention further relates to use of a substance delivery device according to any of the embodiments disclosed herein and/or a delivery assembly according to any of the embodiments disclosed herein for deep delivery of embryos or semen in the uterine horn of a sow or a gilt.

The use provides benefits similar to those described hereinabove, and may particularly be useful for delivery of embryos and/or semen in sows and/or gilts.

In an embodiment, the use is performed 0-9 days after the onset of oestrus, e.g. determined by observing a standing reflex of the sow.

In an embodiment, the use is performed for deep delivery of embryos in the uterine horn of a sow or a gilt, performed 2-9 days after the onset of oestrus, in particular on day 3-8. This way, embryos may be delivered deep in the uterine horn, where a high pregnancy success rate can be achieved.

In an embodiment, the use is performed for deep delivery of semen in the uterine horn of a sow or a gilt, performed 0-5 days after the onset of oestrus, e.g. determined by observing a standing reflex of the sow, in particular on day 0-4.

The use may also be performed a fixed time after weaning or after withdrawal of hormone oestrus synchronisation therapy, e.g. to cause ovarian follicle growth, or agents to cause ovulation).

In an embodiment, the introduction probe is introduced into the cervix but not advanced through the entire cervix, for example introduced up to ⅔ of the length of the cervix.

It has been found that partial insertion of the introduction probe is sufficient for guiding the delivery catheter through the cervix. Further, by not having to pass through the cranial opening with the introduction probe, the procedure may be performed relatively fast and the risk of damaging the uterus with the introduction probe is reduced.

In particular, the introduction probe may be introduced through the caudal opening but not through the cranial opening of the cervix.

In an embodiment, the tip element is advanced up to at least 50% of the length of the uterine horn, for example at least 75%, such as at least 85%, or up to the tip of the uterine horn. This depth has been found to yield relatively high delivery yields.

In an embodiment, the delivery device is inserted for approximately 50 cm into the reproductive tract, upon which the delivery catheter is further advanced into the uterus, in particular at least 50 cm.

In an embodiment, upon insertion of the introduction probe in the cervix, the delivery catheter is advanced further into the reproductive tract passing the cervix and uterine body, upon which the tip element is rotated to select a uterine horn, upon which the catheter is advanced further to enter the selected uterine horn for delivery the uterine horn through the delivery opening.

The cervix may be passed in a relatively short time, for example within 20-30 seconds. In particular, the introduction tube may be positioned in front of the cranial opening within 20-30 seconds.

In an embodiment, the use comprises the step of determining a position of the tip element with respect to the uterine horn, in particular with respect to the tip of the uterine horn.

The position may be determined using an imaging system, such as an ultrasound or x-ray system. Doppler or pulsed doppler ultrasound may be used. The imaging system may comprise a rectal probe.

In an embodiment, the use comprises the step of retracting the catheter from one of the uterine horns, and orienting the tip element towards the other uterine horn, e.g. by rotating the distal end around the longitudinal axis. The catheter may then be advanced in the other uterine horn, for example in a single procedure without having to pass the cervix again. Thus, delivery may then be repeated in the other uterine horn.

In an embodiment, the use comprises the step of determining the delivery and/or properties of the delivered substance, e.g. by imaging the uterus of the pig using an imaging system. Pregnancy may for example be detected by imaging with ultrasound.

In an embodiment, the use of the delivery device comprises a step of at least partial sterilisation in an autoclave.

The invention further relates to a method for delivery of embryos or semen deep in a reproductive tract-shaped object using a substance delivery device, for example according to any of the embodiments disclosed herein and/or a substance delivery assembly, for example according to any of the embodiments disclosed herein, comprising the steps of:
moving an introduction probe having a guide lumen partially into the reproductive tract-shaped object in a movement direction;
moving a delivery catheter arranged in the guide lumen to slide within the guide lumen in the movement direction;
rotating the delivery catheter, which may have a length a length of from a proximal end up to a delivery opening of at least 1.7 m, with respect to the introduction probe to steer the delivery catheter with a tip element thereof, which may have a bend in a bending direction that is less than 45° in a direction angled to the movement direction;
further moving the delivery catheter at a proximal end in the movement direction, therewith advancing the tip element; and
providing delivery fluid via a delivery opening.

The method may be performed using any steps as described for the embodiments of the use of the substance delivery device as disclosed herein, and therewith provide similar benefits. The method may particularly be useful for delivery of embryos and/or semen in sows and/or gilts.

In an embodiment, the introduction probe is introduced into the cervix but not advanced through the entire cervix, for example introduced up to ⅔ of the length of the cervix.

In an embodiment, the tip element is advanced up to at least 50% of the length of the uterine horn, for example at least 75%, such as at least 85%, or up to the tip of the uterine horn. This depth has been found to yield relatively high delivery yields.

In an embodiment, the length of the delivery catheter from the proximal end up to the delivery opening is at least 1.7 m.

### BRIEF DESCRIPTION OF THE DRAWINGS

Further characteristics and advantages of the invention will now be elucidated by a description of embodiments of the invention, with reference to the accompanying drawings, in which:

FIG. 1 schematically depicts an delivery device according to an embodiment of the invention;

FIG. 2A schematically depicts a cross section of the embodiment of the delivery device of FIG. 1, along line A-A of FIG. 1;

FIG. 2B schematically depicts a top view of a delivery section and tip element of the delivery device of FIG. 1;

FIG. 2C schematically depicts a detailed view of section B in FIG. 2B;

FIG. 2D schematically depicts a side view of the delivery section and tip element of FIG. 2B;

FIG. 3A schematically depicts a top view of a tip element and delivery section of the delivery device according to another embodiment;

FIG. 3B schematically depicts a side view of a delivery section of an delivery device according to the embodiment of FIG. 3A, wherein several dimensions are shown as an example;

FIG. 3C schematically depicts a cross section of the embodiment of the delivery section of FIG. 3A, along line B-B of FIG. 3A;

FIG. 3D schematically depicts an elongate element of a delivery section according to an embodiment of the invention;

FIG. 3E schematically depicts an elongate element of a delivery section according to another embodiment of the invention;

FIG. 4A schematically depicts a reproductive tract of a sow or a gilt;

FIG. 4B schematically depicts the reproductive tract of FIG. 3A, with the insertion tube according of an delivery device according to an embodiment of the invention inserted therein;

FIG. 4C schematically depicts the delivery device of FIG. 3B, wherein the delivery catheter is inserted further into the uterus;

FIG. 4D schematically depicts the delivery device of FIG. 3C, upon rotation of the delivery catheter with respect to the insertion tube to steer the delivery catheter into a selected uterine horn;

FIG. 4E schematically depicts the delivery device of FIG. 3D, wherein the delivery catheter is advanced further into the selected uterine horn;

FIG. 5A schematically depicts bottom view of a delivery section according to an embodiment;

FIG. 5B schematically depicts an enlarged perspective view of the connector of the delivery section of FIG. 5A;

FIG. 5C schematically depicts a side perspective view of the connector of FIG. 5B;

FIG. 6A schematically depicts a perspective view of a delivery section connector according to another embodiment;

FIG. 6B schematically depicts a side view of the delivery section connector of FIG. 6A;

FIG. 7A shows an X-ray image of an insemination procedure being performed in a pig using a conventional catheter;

FIG. 7B shows an X-ray image of the insemination procedure of FIG. 7A being performed using a catheter according to the invention.

Throughout the figures, the same reference numerals are used to refer to corresponding components or to components that have a corresponding function.

### DETAILED DESCRIPTION OF EMBODIMENTS

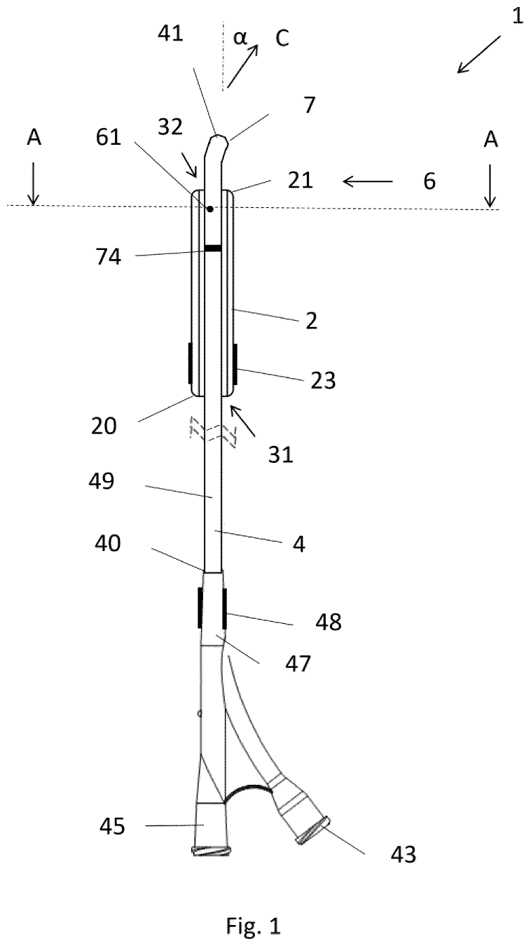

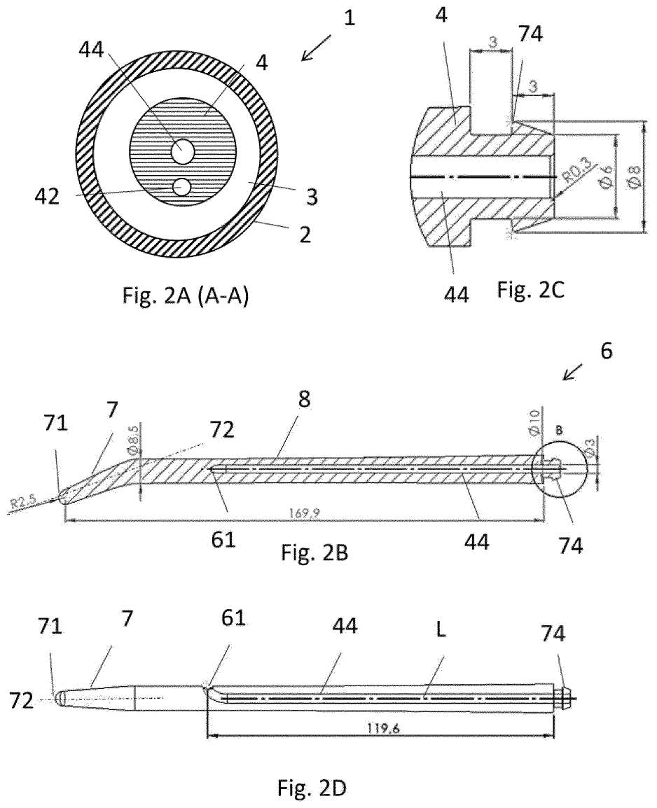

FIGS. 1, 2A, 2B, 2C and 2D schematically depict an delivery device 1 according to an embodiment of the invention. All dimensions in the figures are shown as an example. The delivery device 1 comprises an introduction probe 2 to be partially introduced into the cervix of the pig, provided with a guide lumen 3;

The delivery device 1 further comprises a delivery catheter 4, slidably arranged in the guide lumen 3. The delivery catheter 4 comprises a proximal end 40 and a distal end 41, and a delivery lumen 44 extending from an inlet port 45 at or near the proximal end 40. At the proximal end 40, a manifold 47 is provided having the inlet port 45.

The introduction probe 2 has a proximal end 20 and a distal end 21. The introduction probe 2 is a relatively rigid, straight tube provided with a flat outer surface, having a length of at least 30 cm, in particular 35 cm.

The guide lumen 3 extends from a proximal opening 31 in the proximal end 20 up to a distal catheter opening 32 in the distal end 21. The introduction probe 2 has, towards the distal end 41, a decreasing outer diameter and a rounded end surface 21. The introduction probe 2 is relatively stiff and configured to be positioned, from the perspective of an operator, in front of the caudal opening of the cervix. The proximal end 20 comprises a grip 23. Further, the manifold 47 of the delivery catheter 4 is provided with a grip 48.

The delivery catheter 4 is configured to extend through the distal opening 32 in communication with the guide lumen 3 at the distal end 21 of the introduction probe 2.

The delivery catheter 4 comprises a delivery section 6 arranged at or near the distal end 41 to be introduced inside the uterine horn. The delivery section 6 comprises a delivery openings 61. The delivery opening 61 is fluidly connected to the delivery lumen 44 and has a diameter of equal to a diameter of the delivery lumen 44, which is at least 1 mm, e.g. 1-6 mm, in particular 2.5 mm in this embodiment.

The delivery catheter 4 comprises a tip element 7 arranged at or near the distal end 41. The tip element 41 is shaped to steer the delivery catheter 4. The delivery catheter 4 is configured to be advanced through the cranial opening of the cervix when the introduction tube 2 is inserted partially into the cervix and not through the cranial opening of the cervix. The tip element 7 comprises a bend (a) in a bending direction C that is less than 45° to steer the delivery catheter 4. The delivery opening 61 is positioned with respect to the bending direction C to allow fluid flow out of the delivery opening 61. In this embodiment, the delivery opening is positioned on the side of the catheter 41, but may also be located on another location on the outer surface, e.g. having a position rotated about the longitudinal axis L of the catheter 41.

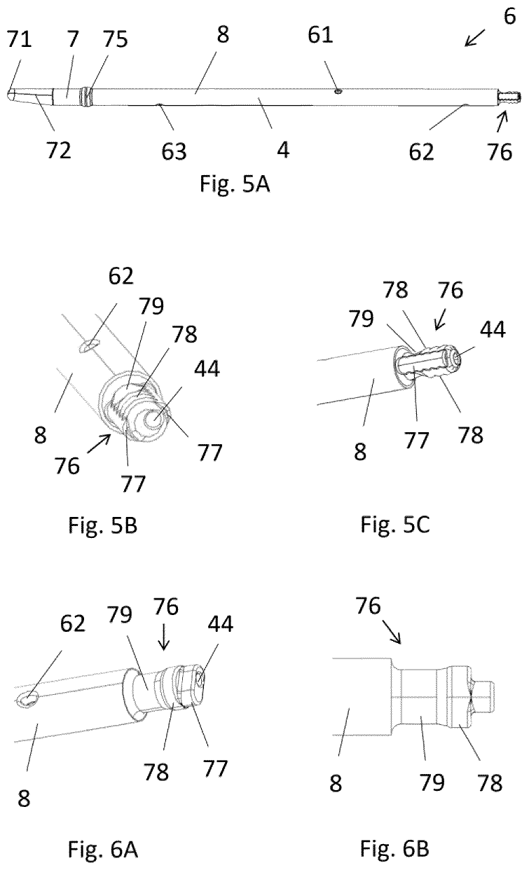

The delivery catheter 4 comprises an uteral section 49 and the tip element 7 and the delivery section 6 are a single distinct element coupled to the uteral section 49. The tip element 7 and/or the delivery section 6 are detachably coupled to the rest of the catheter, i.e. the uteral section 49, via a detachable connection, such as a push-fit connection 74 or threaded connection 74′. As such, the delivery section 6 in this embodiment is detachable from the uteral section 49 of the catheter. Instead of a threaded connection 74, other connections types may be used, such as the connectors 76 depicted in FIGS. 5A-C and 6A-B. Although the connectors 76 are depicted in an embodiment having multiple openings 61, 62, 63 and a collar 75, the connectors 76, the multiple openings 61, 62, 63 and the collar 75 may be provided separately in an embodiment.

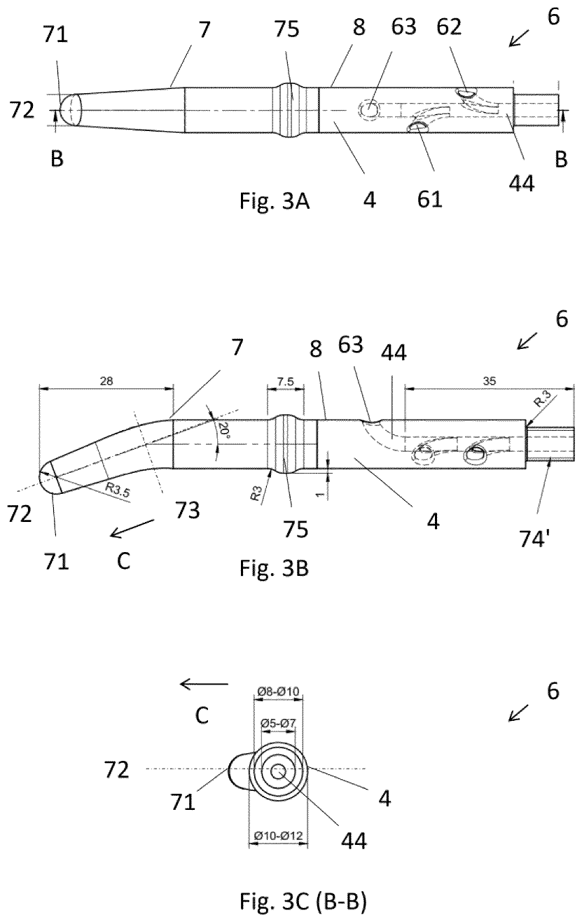

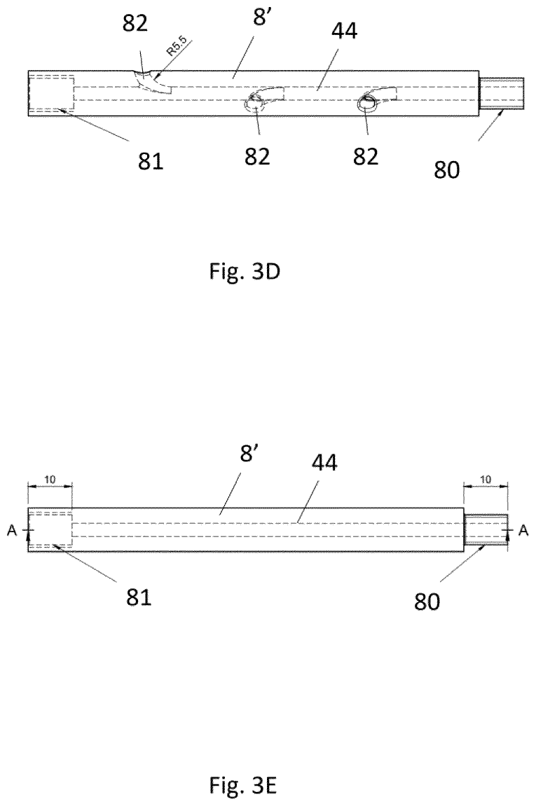

As shown in the embodiments of FIGS. 1-3, the tip element 7 is a bent rounded cone, in particular an oblique cone having a rounded top 71 at the distal end 41 that is bent having the cone axis 72 in a first bending direction C at an angle that is less than 45°, in particular 6°-8°, for example 8°, with respect to the longitudinal axis L of the delivery section 6. In FIG. 3B, the bend angle is 20°.

The top 71 may also be biased in a second bending direction, such that a projection of the top 71 on a cross sectional base plane 73 is not located in the centre of the base plane 73. The second bending direction may coincide with the first bending direction C. The tip element 7 may be bent in the second bending direction. As an example only, dimensions (in mm) are shown in FIG. 3B-3E. In FIGS. 1-2, no collar 75 is provided around the circumference of the catheter 4 between the tip element 7 and the delivery opening 61, and the catheter has a smooth outer surface between the tip element 7 and the delivery opening 61 free from outer protrusions between the tip element 7 and the guide lumen 32, when arranged therein, as depicted in FIG. 1.

A distance between the tip element and the delivery opening is less than 50 cm, such as less than 30 cm, in particular less than 10 cm.

The delivery catheter 4 may be provided with additional lumina 42, 46 (not shown in FIGS. 1, 3A-3E) fluidly connected to additional side ports (43). The delivery opening may be located in the tip element such that delivery fluid can be provided near the distal end 41.

The delivery catheter 4 has a relatively high torsional stiffness to allow for rotation around a longitudinal axis such that the tip element 7 may be rotated by rotating a proximal end 40 of the catheter 4 and that a uterine horn may be selected by rotation of the tip element 7 from the proximal end 40. The delivery catheter 4 has relatively low bending stiffness for following the shape of the uterine horn and a relatively high longitudinal stiffness.

The delivery section 6 comprises an elongate element 8 that spaces the tip element 7 from the uteral section 49 along the longitudinal axis, such that a length from the tip element 7 to the uteral section is 5-70 cm, for example 5-50 cm. The delivery opening 61 is provided in the elongate element 8.

However, for adapting the distance between the tip element and the uteral section 49, additional elongate elements 8′ may be provided, as shown in FIGS. 3D and 3E. As an example only, dimensions (in mm) are shown in FIGS. 3D and 3E. The additional elongate element 8′ may have additional openings 82 and may be provided with connections, such as threaded connections 80, 81 corresponding to the connections, e.g. threaded connections 74, of the delivery section 6, tip element 7 and/or other parts of the delivery catheter 4 for fluidly connecting the additional openings 82 to the delivery lumen 44. Other examples of connections are depicted in FIGS. 5B-C and 6A-B.

The tip element 7 is less rigid than the elongate element 8. The elongate element 8 spaces the tip element 7 from the uteral section 49 in the longitudinal direction L, such that wherein a length from the tip element 7 to the uteral section 49 is 5-70 cm, for example 10-50 cm. A length of the delivery catheter from the proximal end up to the delivery opening is at least 1.7 m, in particular, 170 to 345 cm. The length may also be at least 2.0 meter, for example at least 2.5 meter.

An outer diameter of the delivery catheter 4 is at least 1 mm, for example 2 mm.

The tip element 7 is autoclave-resistant for at least 15, such as 30, minutes at 121° C., and/or at least 3, such as 6, minutes at 134° C., for example at a steam pressure of 2-5 bar.

The delivery section 6 including the tip element 7 and the delivery lumen 44 have a smooth surface. The delivery catheter 2 comprises a silicone rubber and the tip element is made of a 3d-printed silicone rubber having an elastic modulus of 2.5-7.5 N/mm², such as TrueSil A50. The tip element and the delivery section comprise a material having an A classification of A35-A100, such as A50-A100. The tip element 7 is connected to the elongate element 8 at collar 75 via a silicone glue, such as Dow Corning 782.

The delivery assembly 1 may comprise an imaging system such as an ultrasound or pulsating doppler system, for imaging a position of the tip element with respect to the uterine horn.

An injection device, such as a syringe or pump may be provided, that is fluidly connectable to the delivery lumen via the inlet port 45 for injecting substance into the uterine horn through the delivery opening 45. Further, an injectable substance container, e.g. a fluid container may be provided, associated with the injection device and fluidly connectable to the delivery lumen via the injection device for deep delivery of the substance in the uterine horn. The substance container may also be provided in the injection device, e.g. as a fluid chamber in a syringe. The volume of the substance container may be less than 600 μL. The injection device may comprise transport fluid injection means and/or the substance container may comprise a second fluid, such that the injection device is configured to inject the deliverable fluid by injecting a transport fluid before and/or after the substance.

Another embodiment of a delivery section 6 is depicted in FIG. 5A. Other properties may be similar to the delivery section 6 of FIG. 3A. The delivery sections comprises a connector 76, which is depicted in FIGS. 5B and 5C. Another possible connector 76 is depicted in FIGS. 6A and 6B. Complementary connectors may be arranged on additional elongate elements 8′. and/or on the uteral section 49.

The connector 76 comprises radial protrusions 77 that are arranged on the delivery section in a non-rotationally symmetrical way, such that rotation forces may be transferred reliably via the connector 76. The radial protrusions 77 provide the connector with a non-circular cross sectional shape, in particular an oval.

The advantage of the connector 76 of FIGS. 5A-5C and 6A-6B is that no screw thread may be necessary for attachment, allowing a wall thickness of the catheter to be relatively large causing a relatively large transversal strength of the connector 76. Additionally or alternatively, a glue, a friction lock, snap fit or other connection is used. For example, an indent 79 may be provided having a first diameter and one or more ridges 78 may be provided having a second diameter larger than the first diameter, such that the connector 76 may be held via glue, friction with the one or more ridges 78 and/or snapping around indent 79 behind a ridge 78.

In the embodiment of FIG. 3, the delivery catheter 4 comprises a collar 75 having an outer diameter that is larger than a diameter of the distal catheter opening 32. Additionally or alternatively, the introduction probe 2 may comprise a holding element to hold the delivery catheter 2. The collar may enhance feeling of forces between the catheter 4 and the reproductive tract.

The catheter 4 may also comprise an inflatable cuff 5 fluidly connected to an inflation lumen, for example the additional lumen 43 via a side port 51, to be inflated for at least partially closing the uterine horn at a proximal end thereof.

The delivery section 6 comprises multiple delivery openings 61, 62, 63 evenly distributed over the length of the delivery section 6 and evenly spaced around the circumference thereof.

The delivery openings 61, 62, 63 are fluidly connected to the delivery lumen 44 and have a diameter of equal to a diameter of the delivery lumen 44, which is 2.5 mm in this embodiment.

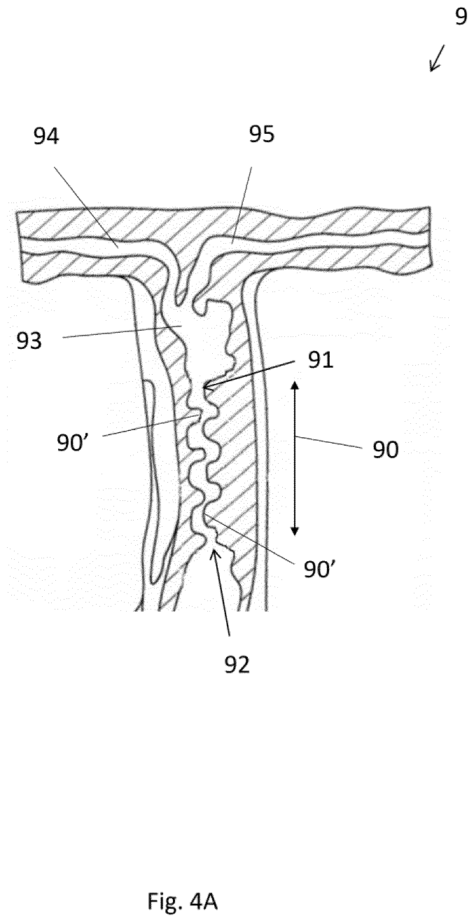

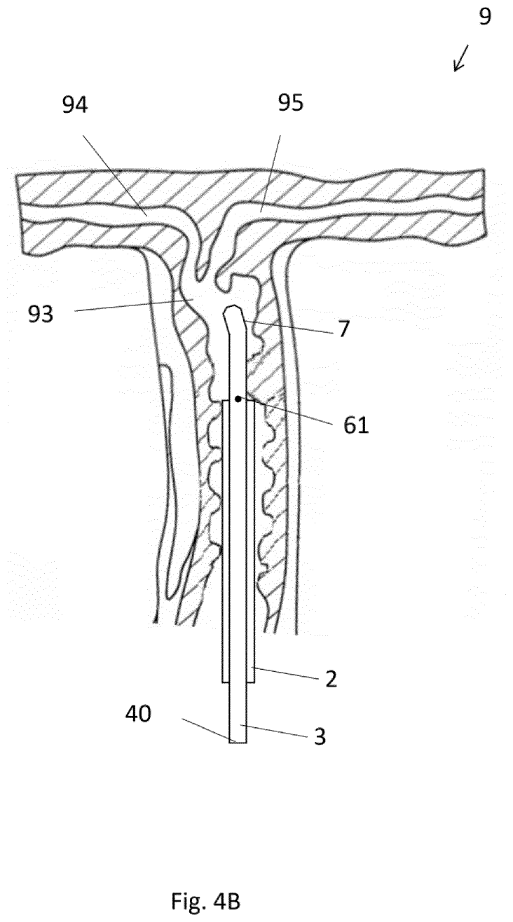

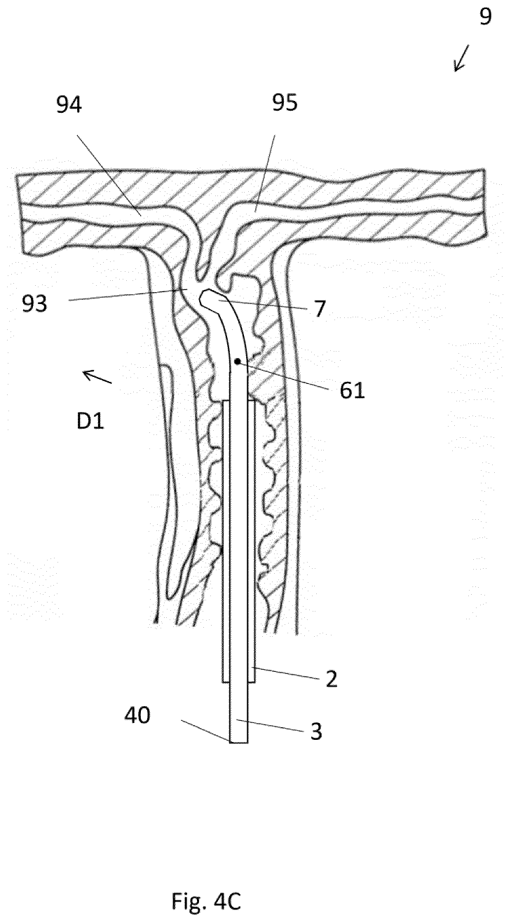

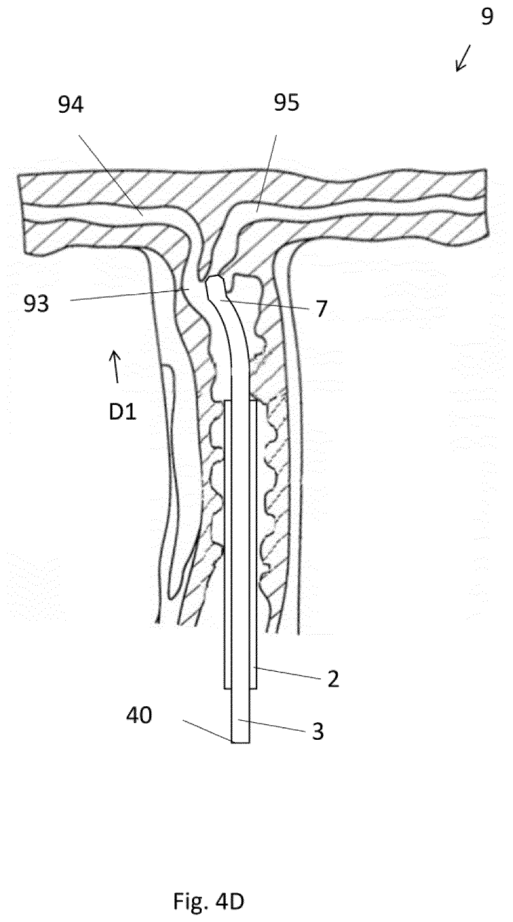

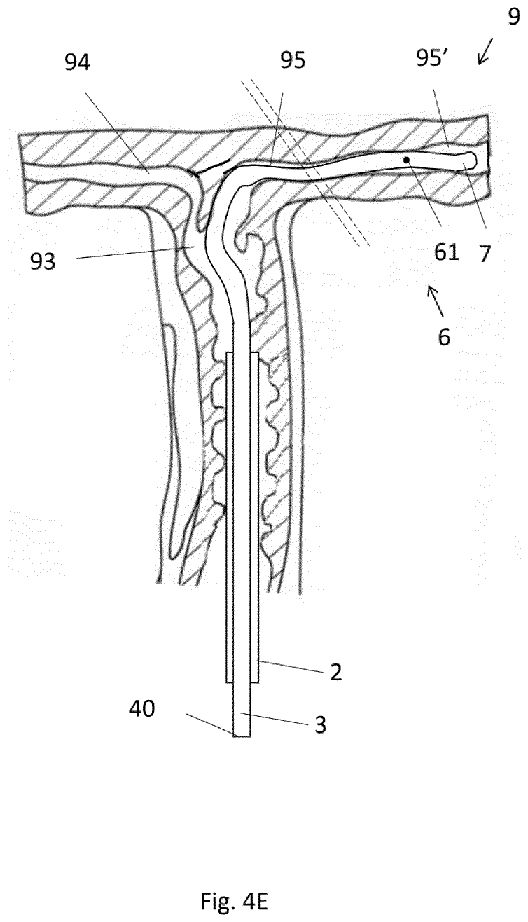

FIGS. 4A-4E schematically show the use of an delivery device according to an embodiment of the invention for delivering embryos and/or semen deep in the uterine horn of a sow or a gilt. In a similar way, other substances may be delivered.

The reproductive tract 9 of a sow or a gilt comprises a cervical canal 90 extending between the relatively narrow caudal opening 92 and cranial opening 91. The cervical canal 90 comprises cervical folds 90′.

Using the delivery device 1, the introduction probe 2 may be inserted partially into the cervical canal 90, for example through the caudal opening 92 but not through the cranial opening 91 as an operator can feel the position of the introduction probe 2 with respect to the cervical tissue. The delivery device 1, is, via the introduction probe 2, first inserted for approximately 50 cm into the reproductive tract.

The cervix may be passed in a relatively short time, for example within 20-30 seconds. In particular, the introduction tube 2 may be positioned in front of the cranial opening 91 within 20-30 seconds.

Upon insertion of the introduction probe 2 in the cervical canal 90, the delivery catheter 2 may be advanced further for at least 50 cm to extend out of the introduction probe 2 through the distal catheter opening 32 into the reproductive tract passing the cervix and uterine body as shown in FIGS. 4B and 4C, without being supported by a guide wire and without being supported by the introduction tube 2 through the entire cervix.

Due to the longitudinal stiffness of the tip element 7, a position thereof with respect to the uterine horns 94, 95 may be determined haptically by an operator. Alternatively, a position may be determined using an imaging system.

Then, by orienting the tip element 7 by rotating distal end 40 around its longitudinal axis, a position of the rounded cone with respect to uterine horns 94, 95 may be selected, as depicted in FIG. 4D. This way, the first bending direction C and/or second bending direction defined by the tip element 7 may rotated to coincide with an opening of one of the uterine horns 94, 95.

The tip element 7 may be advanced into a selected uterine horn 95 and, by means of the bending direction C, is configured to steer the delivery catheter 3 into and through the selected uterine horn 95. The delivery catheter 4 may be positioned to perform delivery at a desired depth 95′ in the uterine horn, for example deep in the uterine horn.

As shown in FIG. 4E, upon advancing the delivery catheter 3 into the selected uterine horn 95 towards a desired depth, delivery of the substance at the desired depth 95′ may be performed by providing an amount of delivery fluid through a inlet port, for example using a syringe or pump, to inject the substance, e.g. fluid containing embryos and/or semen, through the delivery lumen and the delivery opening 61 of the delivery section 6. A delivery volume of less than 600 μL is used.

Upon delivery, the catheter may be retracted from the selected uterine horn, and, optionally, through orienting the tip element towards the other uterine horn, e.g. by rotating distal end 40 around the longitudinal axis, delivery may be repeated in the other uterine horn.

Delivery may be performed 0-9 days after observing a standing reflex of the sow, in particular on day 3-8 for embryo delivery and on day 0-3 for semen delivery. The presence and/or properties of the embryos may be determined, in particular blastocysts, e.g. by imaging the ovaries of the pig using an imaging system. Ovulation may for example be detected by imaging the ovaries with ultrasound.

The delivery device may at least be partially sterilised in an autoclave.

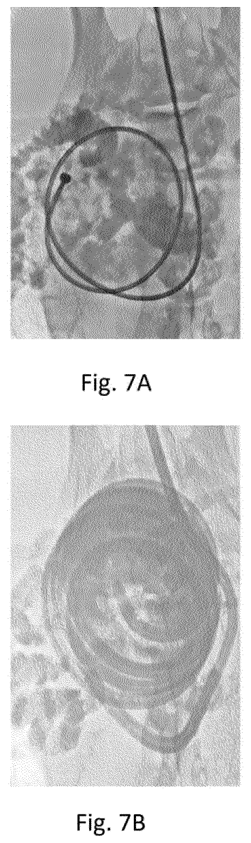

X-Ray images of the procedure are shown in FIGS. 7A and 7B. As can be seen in the figures, using the same procedure as described above, the catheter according to the invention is able to reach much deeper in the uterine horn compared to a catheter of the prior art, which gets stuck in the uterine horn.

1.-26. (canceled)

27. A substance delivery device for deep delivery of embryos or semen in the uterine horn of a pig, in particular a sow or a gilt, comprising:
an introduction probe to be partially introduced into the cervix of the pig, provided with a guide lumen; and
a delivery catheter, slidably arranged in the guide lumen, comprising:
a proximal end and a distal end, an inlet port arranged near the proximal end and a delivery lumen extending from the inlet port towards the distal end;
a delivery section arranged at or near the distal end to be introduced inside the uterine horn, comprising a delivery opening fluidly connected to the delivery lumen; and
a tip element, arranged at the distal end,
wherein the tip element comprises a bend in a bending direction that is less than 45° to steer the delivery catheter,
wherein the delivery opening is positioned with respect to the bending direction to allow fluid flow out of the delivery opening,
wherein a length of the delivery catheter from the proximal end up to the delivery opening is at least 1.7 m, such that upon insertion of the introduction probe in the cervix, the delivery catheter may be advanced further into the reproductive tract passing the cervix and uterine body and entering the uterine horn for delivery of embryos and/or semen into the uterine horn through the delivery opening, and
wherein the tip element is a rounded cone.

28. The substance delivery device according to claim 27, wherein the tip element is a bent rounded cone.

29. The substance delivery device according to claim 28, wherein the tip element comprises a bend having an angle of 2°-30°, for example a bend having an angle of 6-8°.

30. The substance delivery device according to claim 27, wherein the tip element is made from a silicone rubber.

31. The substance delivery device according to claim 27, wherein no collar is provided around the circumference of the catheter between the tip element and the delivery opening, and
wherein the catheter has a smooth outer surface between the tip element and the delivery opening.

32. The substance delivery device according to claim 31, wherein the delivery catheter, between the tip element and the guide lumen, is free from outer protrusions including collars and/or cuffs.

33. The substance delivery device according to claim 27, wherein a distance between the tip element and the delivery opening is less than 50 cm.

34. The substance delivery device according to claim 27, wherein the delivery catheter comprises an uteral section, and
wherein the tip element and/or the delivery section are distinct elements coupled to the uteral section.

35. The substance delivery device according to claim 27, wherein the tip element and/or the delivery section are detachably coupled to the uteral section.

36. The substance delivery device according to claim 27, wherein the tip element has an elastic modulus of 2.5-7.5 N/mm².

37. The substance delivery device according to claim 27, wherein the tip element comprises a material having an A classification of A35-A100 including A50-A100.

38. The substance delivery device according to claim 27, wherein the delivery section comprises a material having an A classification of A35-A100 including A50-A100.

39. The substance delivery device according to claim 27, wherein a diameter of the delivery lumen is at least 1 mm, for example 1-2.5 mm.

40. The substance delivery device according to claim 27, wherein the delivery section comprises an elongate element that spaces the tip element from the uteral section in a longitudinal direction, and
wherein a length from the tip element to the uteral section is 5-70 cm, for example 10-50 cm.

41. The substance delivery device according to claim 27, wherein a length of the delivery catheter from the proximal end up to the delivery opening is in the range of 170 to 345 cm.

42. The substance delivery device according to claim 27, wherein the introduction probe is straight, and/or
wherein the introduction probe has a flat outer surface.

43. A substance delivery assembly, comprising:
the substance delivery device according to claim 27;
an injection device, such as a syringe or pump, fluidly connectable to the delivery lumen via the inlet port for injecting substance in the uterine horn through the delivery opening; and
an injectable substance container, associated with the injection device and fluidly connectable to the delivery lumen for deep delivery of embryos or semen in the uterine horn.

44. The substance delivery assembly according to claim 43, wherein the injectable substance container is a fluid container which comprises a deliverable fluid,
wherein the deliverable fluid comprises embryos and/or semen, and
wherein the volume of the deliverable fluid is less than 600 μL.

45. The substance delivery assembly according to claim 43, wherein the injection device is configured to inject the deliverable substance by injecting a transport fluid before and/or after the substance.

46. A use of the substance delivery device according to claim 42 for deep delivery of embryos or semen in the uterine horn of a sow or a gilt.

47. The use according to claim 46, performed 0-9 days after the onset of oestrus, determined by observing a standing reflex of the sow.

48. The use according to claim 46 for deep delivery of embryos in the uterine horn of a sow or a gilt, performed 2-9 days after the onset of oestrus, in particular on day 3-8.

49. The use according to claim 46 for deep delivery of semen in the uterine horn of a sow or a gilt, performed 0-5 days after the onset of oestrus, determined by observing a standing reflex of the sow, and on day 0-4.

50. The use according to claim 46, wherein the introduction probe is introduced into the cervix but not advanced through the entire cervix, for example introduced up to ⅔ of the length of the cervix.

51. The use according to claim 46, wherein the tip element is advanced up to at least 75%, icnluding at least 85%, of the uterine horn, and up to the tip of the uterine horn.

52. A method for delivery of embryos or semen deep in a reproductive tract-shaped object using a substance delivery device, for example according to claim 42, comprising the steps of:
moving an introduction probe having a guide lumen partially into the reproductive tract-shaped object in a movement direction;
moving a delivery catheter arranged in the guide lumen to slide within the guide lumen in the movement direction;
rotating the delivery catheter with respect to the introduction probe to steer the delivery catheter with a tip element thereof, in a direction angled to the movement direction;
further moving the delivery catheter at a proximal end in the movement direction, therewith advancing the tip element; and
providing delivery fluid via a delivery opening.
